(19) United States
(12) Patent Application Publication
(10) Pub. No.: US 2022/0406097 A1
(43) Pub. Date: Dec. 22, 2022

(54) METHOD FOR QUEUING AND THE RELATED ELECTRONIC DEVICE

(71) Applicant: TAIPEI MEDICAL TECHNOLOGY CO., LTD., TAIPEI CITY (TW)

(72) Inventors: Ching-Chuan HUNG, TAIPEI CITY (TW); Shun Chi WU, TAIPEI CITY (TW); Wan Ting TSENG, TAIPEI CITY (TW); Kuang Lung KO, TAIPEI CITY (TW); Yi Ting TSAI, TAIPEI CITY (TW)

(21) Appl. No.: 17/844,753

(22) Filed: Jun. 21, 2022

(30) Foreign Application Priority Data
Jun. 22, 2021 (TW) 110122820

(51) Int. Cl. G07C 1/10

(52) U.S. Cl. CPC G07C 1/10; G07C 2011/04

(57) ABSTRACT

The present disclosure provides a method for queuing and a related electronic device. The method for queuing in the present disclosure includes: receiving user data of a user; receiving a current serial number and a quantity of queued people of a service station; determining a queuing time based on the current serial number, the quantity of queued people, and a serial number of the user data; receiving a quantity of waiting people of a first stop in response to a first demand value of the user data being true; determining a first waiting time based on the quantity of waiting people of the first stop; and transmitting a first signal of going to the first stop to the user, and queuing the user in a queue of the service station and a queue of the first stop, in response to the first waiting time being less than the queuing time.

### BACKGROUND

### 1. Technical Field

The present disclosure relates to a method for queuing and a related electronic device. More specifically, the present disclosure relates to a method for queuing an order of a user accepting a service and a related electronic device.

### 2. Description of the Related Art

Organizations such as clinics, hospitals, banks, or transport motor vehicles offices for providing services usually allocate different serial numbers to users and provide a service to each user according to an order of the serial numbers.

### SUMMARY

In most cases, before accepting a service, a user may wait a long time. In some cases, when the user obtains a service of a first service station after a long wait, the first service station notifies the user that the user needs to first go to a second service station to obtain another service before obtaining the service of the first service station. For example, when a driver replaces a driving license at a transport motor vehicles office and may finally obtain a driving license replacement service after a wait, a service staff for driving license replacement tells the driver to first go to a ticket inquiry service to query whether there is an outstanding fine before applying to replace the driving license. Both the driver and the service staff have spent too much time.

For example, when a pregnant woman has a prenatal checkup and may enter a consulting room to see a doctor after a long wait, the doctor may tell the pregnant woman to first perform a blood examination or an ultrasound scan. After arriving at a bloodletting counter or an ultrasound department, the pregnant woman needs to wait again for detection. After obtaining a blood report or an ultrasound report, the pregnant woman may need to wait for seeing the doctor again. Both the pregnant woman and the hospital staff have spent too much time.

The present disclosure provides a novel queuing method and a related electronic device. The present disclosure provides a user-oriented queuing method and a related electronic device. In some embodiments, the present disclosure provides a method for queuing. The method includes: receiving user data of a user; receiving a current serial number and a quantity of queued people of a service station; determining a queuing time based on the current serial number, the quantity of queued people, and a serial number of the user data; receiving a quantity of waiting people of a first stop in response to a first demand value of the user data being true; determining a first waiting time based on the quantity of waiting people of the first stop; and transmitting a first signal of going to the first stop to the user, and queuing the user in a queue of the service station and a queue of the first stop, in response to the first waiting time being less than the queuing time.

In some embodiments, the present disclosure provides an electronic device. The electronic device includes a communication module, a memory module, and a processing module. The communication module is configured to be communicatively coupled to a user device of a user. The communication module is configured to: receive user data of the user; receive a current serial number and a quantity of queued people of a service station; receive a quantity of waiting people of a first stop in response to a first demand value of the user data being true; transmit a first signal of going to the first stop to the user device in response to a first waiting time being less than a queuing time. The memory module is configured to store a plurality of instructions and information. The processing module is configured to be coupled to the communication module and the memory module and perform the following operations based on the instructions and the information stored in the memory module: determining the queuing time based on the current serial number of the service station, the quantity of queued people of the service station, and a serial number of the user data; determining the first waiting time based on the quantity of waiting people of the first stop in response to the first demand value of the user data being true; and queuing the user in a queue of the service station and a queue of the first stop in response to the first waiting time being less than the queuing time.

### BRIEF DESCRIPTION OF THE DRAWINGS

FIG. 1 shows a system and an apparatus according to some embodiments of the present disclosure.

FIG. 2 shows a flowchart of a method according to some embodiments of the present disclosure.

FIG. 3A and FIG. 3B show flowcharts of processes according to some embodiments of the present disclosure.

FIG. 4 shows a flowchart of a process according to some embodiments of the present disclosure.

FIG. 5 shows a flowchart of a process according to some embodiments of the present disclosure.

FIG. 6 shows a flowchart of a process according to some embodiments of the present disclosure.

FIG. 7 shows a flowchart of a process according to some embodiments of the present disclosure.

FIG. 8 shows a flowchart of a process according to some embodiments of the present disclosure.

FIG. 9 shows a flowchart of a process according to some embodiments of the present disclosure.

FIG. 10 shows a flowchart of a process according to some embodiments of the present disclosure.

To better understand the aspects of the present disclosure and additional aspects and embodiments thereof, reference is made to the following implementations in conjunction with the accompanying drawings. In the accompanying drawings, similar reference symbols indicate similar elements. It should be noted that various features may not be drawn to scale. Actually, the sizes of the various features may be increased or reduced arbitrarily for the purpose of clear description.

### DETAILED DESCRIPTIONS

The method, the system, and other aspects of the present disclosure are described. References will be made to some embodiments of the present disclosure, and examples of the embodiments will be described in the accompanying drawings. Although the present disclosure is described with reference to the embodiments, it will be understood that the present disclosure is not intended to be limited to these specific embodiments. On the contrary, the present disclosure intends to cover alternative solutions, modifications, and equivalents that fall within the spirit and scope of the present disclosure. Therefore, the specification and the accompanying drawings should be understood illustratively but not restrictively.

In addition, a plurality of specific details are described in the following descriptions to provide further understanding of the present disclosure. However, a person of ordinary skill in the art may implement the present disclosure without the specific details. In other cases, in order to avoid obscuring the aspects of the present disclosure, methods, processes, operations, components, and networks well known to a person of ordinary skill in the art have not been described in detail.

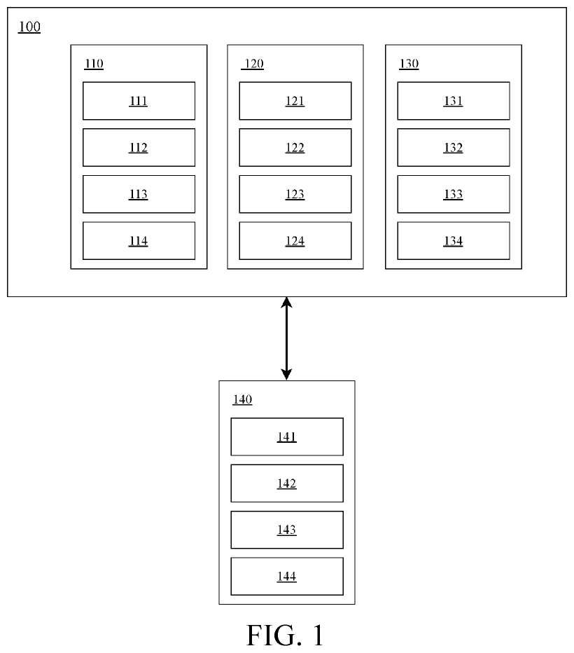

FIG. 1 shows a system 100 and a user device 140 according to some embodiments of the present disclosure. The system 100 may include a server 110, a database 120, and a service station apparatus 130. In some embodiments, the server 110 may be arranged in a hospital, a clinic, a bank, or a motor vehicles office. The database 120 may be arranged in a hospital, a clinic, a bank, or a motor vehicles office. The database 120 may be configured to store user data and data of a service station. The service station apparatus 130 may be arranged at a position such as a counter of a hospital or a clinic, a consulting room, a laboratory, an ultrasound department, or an X-ray room. The service station apparatus 130 may be arranged at different service counters of a bank. The service station apparatus 130 may be arranged at different service counters of a motor vehicles office. The service station apparatus 130 may be used as a terminal for a service staff to use.

The server 110 may include an input/output module 111, a memory module 112, a processing module 113, and a communication module 114. The database 120 may include an input/output module 121, a memory module 122, a processing module 123, and a communication module 124. The service station apparatus 130 may include an input/output module 131, a memory module 132, a processing module 133, and a communication module 134. The user device 140 may include an input/output module 141, a memory module 142, a processing module 143, and a communication module 144.

The server 110 may be a working station or a computer. The database 120 may be a working station or a computer. In some embodiments, the database 120 may be integrated into the server 110. The service station apparatus 130 may be a computer, a personal digital assistant, a smailphone, or a tablet computer. The user device 140 may be a computer, a personal digital assistant, a smailphone, or a tablet computer.

The server 110, the database 120, and the service station apparatus 130 may be communicatively coupled to each other in a wired manner, for example, Ethernet, a coaxial cable, a universal serial bus (USB), or another line with a communication function. The server 110, the database 120, and the service station apparatus 130 may be communicatively coupled to each other in a wireless manner, for example, Bluetooth, IEEE 802.11, long term evolution (LTE), 5G, or another wireless communication protocol.

The system 100 may be communicatively coupled to the user device 140 in a wireless manner, for example, Bluetooth, IEEE 802.11, LTE, 5G, or another wireless communication protocol. In some embodiments, the system 100 may be communicatively coupled to the user device 140 in a wired manner, for example, Ethernet, a coaxial cable, a USB, or another line with a communication function. The system 100 may be communicatively coupled to the user device 140 by using the communication module 114 of the server 110. The system 100 may be communicatively coupled to the user device 140 by the communication module 134 of the service station apparatus 130. In some embodiments, the system 100 may be communicatively coupled to the user device 140 by using an additional communication module.

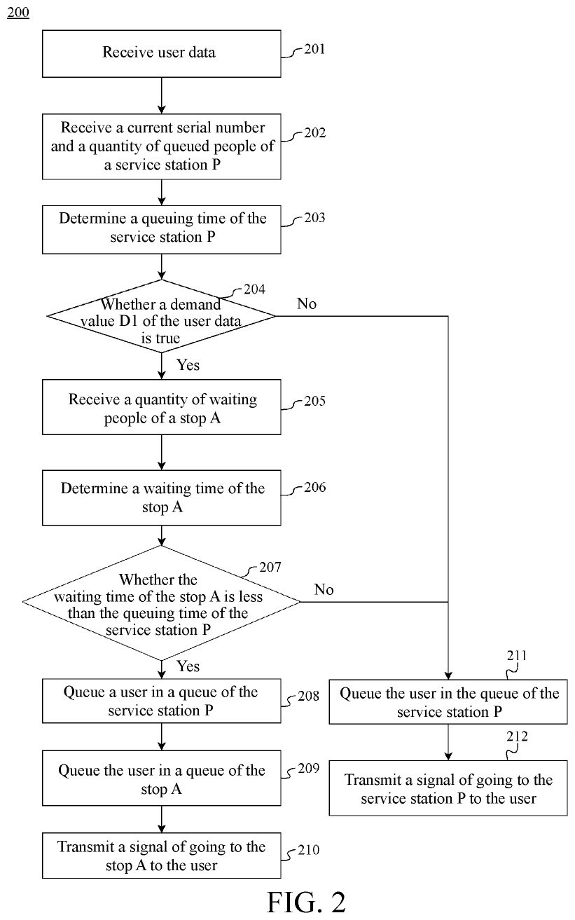

The input/output module 111, the memory module 112, the processing module 113, and the communication module 114 of the server 110 may be configured to perform operations described below from FIG. 2 to FIG. 10. The input/output module 131, the memory module 132, the processing module 133, and the communication module 134 of the service station apparatus 130 may be configured to perform the operations described below from FIG. 2 to FIG. 10. In some embodiments, the system 100 may be configured to perform the operations described below from FIG. 2 to FIG. 10 by using an additional input/output module, memory module, processing module, and communication module.

FIG. 2 shows a flowchart of a process 200 according to some embodiments of the present disclosure. Operations of the process 200 may be performed by the system 100 shown in FIG. 1. The operations of the process 200 may be performed by the server 110 or the service station apparatus 130 in the system 100 shown in FIG. 1. The operations of the process 200 may be performed by the system 100 and the user device 140 shown in FIG. 1 cooperatively. An operation 201 may include: receiving user data. The user data may be received from the database 120. The communication module may be configured to perform the operation 201. In some embodiments, the operation 201 may be performed in a case that a user has checked in an organization such as a hospital, a clinic, a bank, or a motor vehicles office.

An operation 202 may include: receiving a current serial number and a quantity of queued people of a service station P. The current serial number and the quantity of queued people of the service station P may be received from the database 120. The current serial number and the quantity of queued people of the service station P may be received from the service station apparatus 130 of the service station P. The communication module may be configured to perform the operation 202. In some embodiments, the operation 202 may be performed in a case that a user has checked in an organization such as a hospital, a clinic, a bank, or a motor vehicles office. In some embodiments, the current serial number and the quantity of queued people of the service station P may indicate a serial number that is being in a consulting room of a hospital and a quantity of people who have checked in but not seeing a doctor currently in the hospital. In some embodiments, the current serial number and the quantity of queued people of the service station P may indicate a serial number that is currently served by a driving license replacement counter and a quantity of people who have checked in but not been served by the driving license replacement counter.

An operation 203 may include: determining a queuing time of the service station P. The queuing time of the user in the service station P may be determined based on the current serial number of the service station P, the quantity of queued people of the service station P, and a serial number of the user. The serial number of the user may be at a serial number column of the user data. In some embodiments, the serial number of the user data may indicate a reservation serial number of a patient. In some embodiments, the serial number of the user data may indicate a reservation serial number of a driver. The processing module may be configured to perform the operation 203. In some embodiments, the queuing time of the service station P may indicate a time for a patient to wait for seeing a doctor. In some embodiments, the queuing time of the service station P may indicate a time for a driver to wait for replacing a driving license.

An operation 204 may include: determining whether a demand value D1 of a user is true. The demand value D1 of the user may be a column in the user data. The processing module may be configured to perform the operation 204. In some embodiments, the demand value D1 of the user being true may indicate that the patient needs to have an ultrasound scan before seeing a doctor. In some embodiments, the demand value D1 of the user being true may indicate that the driver needs to first pay a fine before replacing the driving license.

If the demand value D1 of the user is true, an operation 205 may be performed. The operation 205 may include: receiving a quantity of waiting people of a stop A. The communication module may be configured to perform the operation 205. In some embodiments, the quantity of waiting people of the stop A may indicate a quantity of people that wait for an ultrasound scan. In some embodiments, the quantity of waiting people of the stop A may indicate a quantity of people that wait for paying a fine.

An operation 206 may include: determining a waiting time of the stop A. The waiting time of the user in the stop A may be determined based on the quantity of waiting people of the stop A. The processing module may be configured to perform the operation 206. In some embodiments, the waiting time of the stop A may indicate a time for the patient to wait for the ultrasound scan. In some embodiments, the waiting time of the stop A may indicate a time for the driver to wait for paying the fine.

An operation 207 may include: determining whether the waiting time of the stop A is less than the queuing time of the service station P. The processing module may be configured to perform the operation 207. In some embodiments, the waiting time of the stop A being less than the queuing time of the service station P may indicate that a waiting time of the ultrasound scan is less than a waiting time of seeing a doctor. In some embodiments, the waiting time of the stop A being less than the queuing time of the service station P may indicate that a waiting time of paying the fine is less than a waiting time of replacing the driving license.

If the waiting time of the stop A is less than the queuing time of the service station P, an operation 208 may be performed. The operation 208 may include: queuing the user in a queue of the service station P. The processing module may be configured to perform the operation 208. In some embodiments, queuing the user in the queue of the service station P may indicate that the patient is queued in a queue of seeing a doctor. In some embodiments, queuing the user in the queue of the service station P may indicate that the driver is queued in a queue of replacing the driving license.

An operation 209 includes: queuing the user in a queue of the stop A. The processing module may be configured to perform the operation 209. In some embodiments, queuing the user in the queue of the stop A may indicate that the user is queued in a queue of performing an ultrasound scan. In some embodiments, queuing the user in the queue of the stop A may indicate that the user is queued in a queue of paying the fine.

An operation 210 includes: transmitting a signal of going to the stop A to the user. The operation 210 includes: transmitting the signal of going to the stop A to the user device 140. The operation 210 includes: transmitting the signal of first going to the stop A and then going to the service station P to the user. The operation 210 includes: transmitting the signal of first going to the stop A and then going to the service station P to the user device 140. The communication module may be configured to perform the operation 210. In some embodiments, transmitting the signal of going to the stop A to the user may indicate that the patient is notified to go to an ultrasound department. In some embodiments, transmitting the signal of going to the stop A to the user may indicate that the driver is notified to go to a fine payment counter.

Referring to the operation 204 again, if the demand value D1 of the user is not true, an operation 211 may be performed. The operation 211 may include: queuing the user in a queue of the service station P. The processing module may be configured to perform the operation 211. In some embodiments, queuing the user in the queue of the service station P may indicate that the patient is queued in a queue of seeing a doctor. In some embodiments, queuing the user in the queue of the service station P may indicate that the driver is queued in a queue of replacing the driving license.

An operation 212 includes: transmitting a signal of going to the service station P to the user. The operation 212 includes: transmitting the signal of going to the service station P to the user device 140. The operation 212 includes: transmitting the signal of first going to the service station P and then going to the stop A to the user. The operation 212 includes: transmitting the signal of first going to the service station P and then going to the stop A to the user device 140. The communication module may be configured to perform the operation 212. In some embodiments, the transmitting the signal of going to the service station P to the user may indicate that the patient is notified to go to a consulting room. In some embodiments, the transmitting the signal of going to the service station P to the user may indicate that the driver is notified to go to a driving license replacement counter.

Referring to the operation 207 again, if the waiting time of the stop A is not less than the queuing time of the service station P, the operation 211 and the operation 212 may be performed.

According to the process 200, one, which has a relatively small waiting time, of pre-work of the stop A or main work of the service station P may be first performed, for example, one of two cases of first performing the pre-work of the stop A and then performing the main work of the service station P, and performing the main work of the service station P. According to the process 200, a waiting time of the pre-work of the stop A and a waiting time of the main work of the service station P may be reduced.

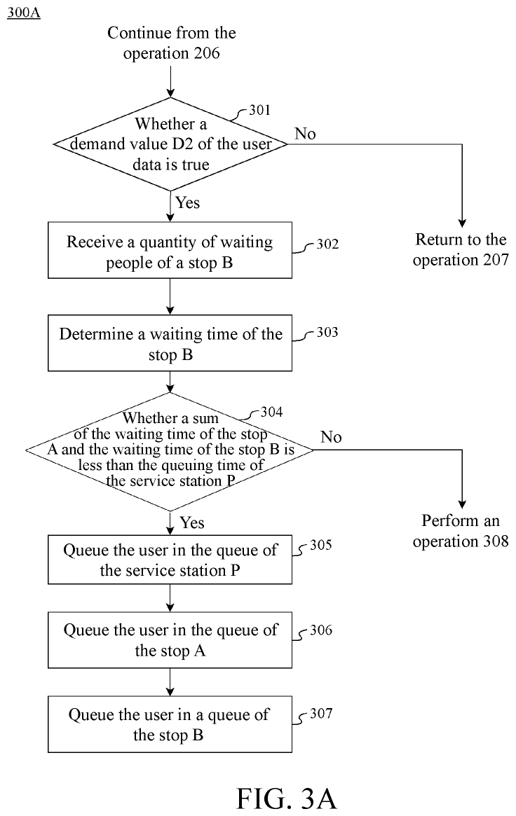

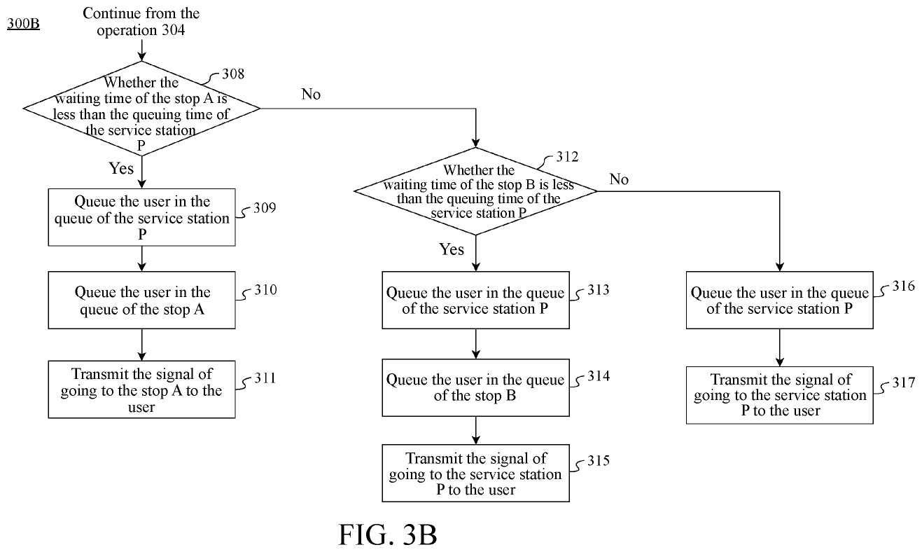

FIG. 3A shows a flowchart of a process 300A according to some embodiments of the present disclosure. Operations of the process 300A may be performed by the system 100 shown in FIG. 1. The operations of the process 300A may be performed by the server 110 or the service station apparatus 130 in the system 100 shown in FIG. 1. The operations of the process 300A may be performed by the system 100 and the user device 140 shown in FIG. 1 cooperatively. The process 300A may be continued from the operation 206. An operation 301 may include: determining whether a demand value D2 of a user is true. The demand value D2 of the user may be a column in the user data. The processing module may be configured to perform the operation 301. In some embodiments, the demand value D2 of the user being true may indicate that a patient needs to first have a blood examination before seeing a doctor. In some embodiments, the demand value D2 of the user being true may indicate that a driver needs to first perform a cognitive function test before replacing the driving license.

If the demand value D2 of the user is true, an operation 302 may be performed. The operation 302 may include: receiving a quantity of waiting people of a stop B. The communication module may be configured to perform the operation 302. In some embodiments, the quantity of waiting people of the stop B may indicate a quantity of people that wait for the blood examination. In some embodiments, the quantity of waiting people of the stop B may indicate a quantity of people that wait for performing the cognitive function test.

An operation 303 may include: determining a waiting time of the stop B. The waiting time of the user in the stop B may be determined based on the quantity of waiting people of the stop B. The processing module may be configured to perform the operation 303. In some embodiments, the waiting time of the stop B may indicate a time for the patient to wait for the blood examination. In some embodiments, the waiting time of the stop B may indicate a time for the driver to wait for performing the cognitive function test.

An operation 304 may include: determining whether a sum of the waiting time of the stop A and the waiting time of the stop B is less than the queuing time of the service station P. The processing module may be configured to perform the operation 304. In some embodiments, the sum of the waiting time of the stop A and the waiting time of the stop B being less than the queuing time of the service station P may indicate that a sum of the waiting time of the ultrasound scan and the waiting time of the blood examination is less than the waiting time of seeing a doctor. In some embodiments, the sum of the waiting time of the stop A and the waiting time of the stop B being less than the queuing time of the service station P may indicate that a sum of the waiting time of paying the fine and the waiting time of performing the cognitive function test is less than the waiting time of replacing the driving license.

If the sum of the waiting time of the stop A and the waiting time of the stop B is less than the queuing time of the service station P, an operation 305 may be performed. The operation 305 may include: queuing the user in a queue of the service station P. The processing module may be configured to perform the operation 305. In some embodiments, queuing the user in the queue of the service station P may indicate that the patient is queued in a queue of seeing a doctor. In some embodiments, queuing the user in the queue of the service station P may indicate that the driver is queued in a queue of replacing the driving license.

An operation 306 includes: queuing the user in a queue of the stop A. The processing module may be configured to perform the operation 306. In some embodiments, queuing the user in the queue of the stop A may indicate that the patient is queued in the queue of performing the ultrasound scan. In some embodiments, queuing the user in the queue of the stop A may indicate that the driver is queued in the queue of paying the fine.

An operation 307 includes: queuing the user in a queue of the stop B. The processing module may be configured to perform the operation 307. In some embodiments, queuing the user in the queue of the stop B may indicate that the patient is queued in a queue of performing the blood examination. In some embodiments, queuing the user in the queue of the stop B may indicate that the driver is queued in a queue of performing the cognitive function test.

Referring to the operation 301 again, if the demand value D2 of the user is not true, an operation 207 may be performed.

Referring to the operation 304 again, if the sum of the waiting time of the stop A and the waiting time of the stop B is not less than the queuing time of the service station P, an operation 308 shown in FIG. 3B may be performed.

FIG. 3B shows a flowchart of a process 300B according to some embodiments of the present disclosure. Operations of the process 300B may be performed by the system 100 shown in FIG. 1. The operations of the process 300B may be performed by the server 110 or the service station apparatus 130 in the system 100 shown in FIG. 1. The operations of the process 300B may be performed by the system 100 and the user device 140 shown in FIG. 1 cooperatively. The process 300B may be continued from the operation 304. An operation 308 may include: determining whether the waiting time of the stop A is less than the queuing time of the service station P. The processing module may be configured to perform the operation 308. In some embodiments, the waiting time of the stop A being less than the queuing time of the service station P may indicate that a waiting time of the ultrasound scan is less than a waiting time of seeing a doctor. In some embodiments, the waiting time of the stop A being less than the queuing time of the service station P may indicate that a waiting time of paying the fine is less than a waiting time of replacing the driving license.

If the waiting time of the stop A is less than the queuing time of the service station P, an operation 309 may be performed. The operation 309 may include: queuing the user in a queue of the service station P. The processing module may be configured to perform the operation 309. In some embodiments, queuing the user in the queue of the service station P may indicate that the patient is queued in a queue of seeing a doctor. In some embodiments, queuing the user in the queue of the service station P may indicate that the driver is queued in a queue of replacing the driving license.

An operation 310 includes: queuing the user in a queue of the stop A. The processing module may be configured to perform the operation 310. In some embodiments, queuing the user in the queue of the stop A may indicate that the patient is queued in the queue of performing the ultrasound scan. In some embodiments, queuing the user in the queue of the stop A may indicate that the driver is queued in the queue of paying the fine.

An operation 311 includes: transmitting a signal of going to the stop A to the user. The operation 311 includes: transmitting the signal of going to the stop A to the user device 140. The operation 311 includes: transmitting the signal of first going to the stop A and then going to the service station P to the user. The operation 311 includes: transmitting the signal of first going to the stop A and then going to the service station P to the user device 140. The communication module may be configured to perform the operation 311. In some embodiments, transmitting the signal of going to the stop A to the user may indicate that the patient is notified to go to an ultrasound department. In some embodiments, transmitting the signal of going to the stop A to the user may indicate that the driver is notified to go to a fine payment counter.

Referring to the operation 308 again, if the waiting time of the stop A is not less than the queuing time of the service station P, an operation 312 may be performed.

The operation 312 may include: determining whether the waiting time of the stop B is less than the queuing time of the service station P. The processing module may be configured to perform the operation 312. In some embodiments, the waiting time of the stop B being less than the queuing time of the service station P may indicate that the waiting time of the blood examination is less than the waiting time of seeing a doctor. In some embodiments, the waiting time of the stop B being less than the queuing time of the service station P may indicate that the waiting time of performing the cognitive function test is less than the waiting time of replacing the driving license.

If the waiting time of the stop B is less than the queuing time of the service station P, an operation 313 may be performed. The operation 313 may include: queuing the user in a queue of the service station P. The processing module may be configured to perform the operation 313. In some embodiments, queuing the user in the queue of the service station P may indicate that the patient is queued in a queue of seeing a doctor. In some embodiments, queuing the user in the queue of the service station P may indicate that the driver is queued in a queue of replacing the driving license.

An operation 314 includes: queuing the user in a queue of the stop B. The processing module may be configured to perform the operation 314. In some embodiments, queuing the user in the queue of the stop B may indicate that the patient is queued in a queue of performing the blood examination. In some embodiments, queuing the user in the queue of the stop B may indicate that the driver is queued in a queue of performing the cognitive function test.

An operation 315 includes: transmitting a signal of going to the stop B to the user. The operation 315 includes: transmitting the signal of going to the stop B to the user device 140. The operation 315 includes: transmitting the signal of first going to the stop B and then going to the service station P to the user. The operation 315 includes: transmitting the signal of first going to the stop B and then going to the service station P to the user device 140. The communication module may be configured to perform the operation 315. In some embodiments, transmitting the signal of going to the stop B to the user may indicate that the patient is notified to go to a bloodletting room. In some embodiments, transmitting the signal of going to the stop B to the user may indicate that the driver is notified to go to a cognitive function test counter.

Referring to the operation 312 again, if the waiting time of the stop B is not less than the queuing time of the service station P, an operation 316 may be performed. The operation 316 may include: queuing the user in a queue of the service station P. The processing module may be configured to perform the operation 316. In some embodiments, queuing the user in the queue of the service station P may indicate that the patient is queued in the queue of seeing a doctor. In some embodiments, queuing the user in the queue of the service station P may indicate that the driver is queued in a queue of replacing the driving license.

An operation 317 includes: transmitting a signal of going to the service station P to the user. The operation 317 includes: transmitting the signal of going to the service station P to the user device 140. The operation 317 includes: transmitting the signal of first going to the service station P and then going to the stop A and/or the stop B to the user. The operation 317 includes: transmitting the signal of first going to the service station P and then going to the stop A and/or the stop B to the user device 140. The communication module may be configured to perform the operation 317. In some embodiments, the transmitting the signal of going to the service station P to the user may indicate that the patient is notified to go to a consulting room. In some embodiments, the transmitting the signal of going to the service station P to the user may indicate that the driver is notified to go to a driving license replacement counter.

According to the process 300A and the process 300B, a combination with a relatively small waiting time may be selected between two pre-work of the stop A and the stop B and the main work of the service station P. For example, the two pre-work of the stop A and the pre-work the stop B are first performed and then the main work of the service station P is performed. For example, the pre-work of the stop A is first performed, then the pre-work of the stop B is performed, and finally the main work of the service station P is performed. For example, the pre-work of the stop B is first performed, then the pre-work of the stop A is performed, and finally the main work of the service station P is performed. For example, the pre-work of the stop A is first performed, and then the main work of the service station P is performed. For example, the pre-work of the stop B is first performed, and then the main work of the service station P is performed. According to the process 300A and the process 300B, the waiting times of the two pre-work of the stop A and the stop B and the waiting time of the main work of the service station P may be reduced. The stop A and the stop B may be configured to provide different services. The stop A and the stop B may be configured to process different services.

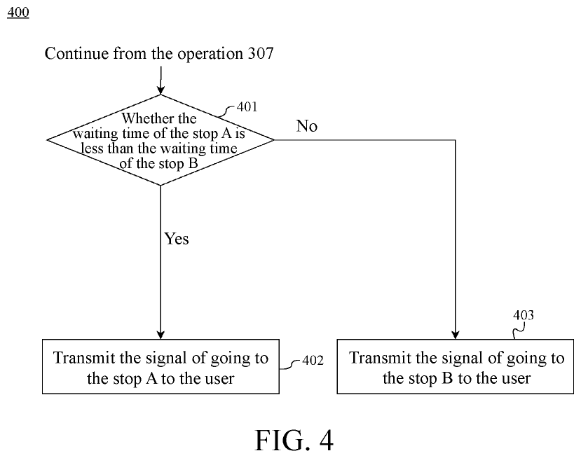

FIG. 4 shows a flowchart of a process 400 according to some embodiments of the present disclosure. Operations of the process 400 may be performed by the system 100 shown in FIG. 1. The operations of the process 400 may be performed by the server 110 or the service station apparatus 130 in the system 100 shown in FIG. 1. The operations of the process 400 may be performed by the system 100 and the user device 140 shown in FIG. 1 cooperatively. The process 400 may be continued from the operation 307. An operation 401 may include: determining whether the waiting time of the stop A is less than the waiting time of the stop B. The processing module may be configured to perform the operation 401. In some embodiments, the waiting time of the stop A being less than the waiting time of the stop B may indicate that the waiting time of performing the ultrasound scan is less than the waiting time of performing the blood examination. In some embodiments, the waiting time of the stop A being less than the waiting time of the stop B may indicate that the waiting time of paying the fine is less than the waiting time of performing the cognitive function test.

If the waiting time of the stop A is less than the waiting time of the stop B, an operation 402 may be performed. An operation 402 includes: transmitting a signal of going to the stop A to the user. The operation 402 includes: transmitting the signal of going to the stop A to the user device 140. The operation 402 includes: transmitting the signal of first going to the stop A and then going to the stop B to the user. The operation 402 includes: transmitting the signal of first going to the stop A and then going to the stop B to the user device 140. The communication module may be configured to perform the operation 402. In some embodiments, transmitting the signal of going to the stop A to the user may indicate that the patient is notified to go to an ultrasound department. In some embodiments, transmitting the signal of going to the stop A to the user may indicate that the driver is notified to go to a fine payment counter.

If the waiting time of the stop A is not less than the waiting time of the stop B, an operation 403 may be performed. An operation 403 includes: transmitting a signal of going to the stop B to the user. The operation 403 includes: transmitting the signal of going to the stop B to the user device 140. The operation 403 includes: transmitting the signal of first going to the stop B and then going to the stop A to the user. The operation 403 includes: transmitting the signal of first going to the stop B and then going to the stop A to the user device 140. The communication module may be configured to perform the operation 403. In some embodiments, transmitting the signal of going to the stop B to the user may indicate that the patient is notified to go to a bloodletting room. In some embodiments, transmitting the signal of going to the stop B to the user may indicate that the driver is notified to go to a cognitive function test counter.

According to the process 400, the user may first perform the pre-work with a relatively small waiting time of the two pre-work of the stop A and the stop B, to reduce the waiting times of the two pre-work. The stop A and the stop B may be configured to provide different services. The stop A and the stop B may be configured to process different services.

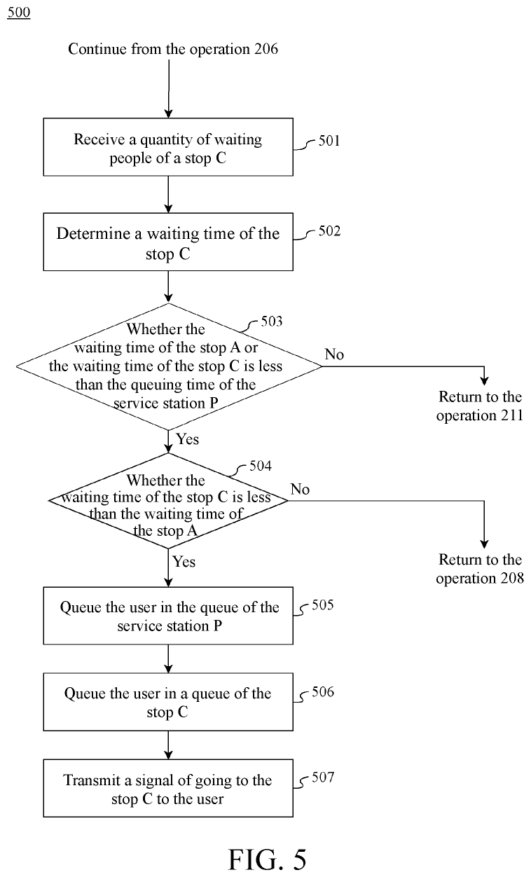

FIG. 5 shows a flowchart of a process 500 according to some embodiments of the present disclosure. Operations of the process 500 may be performed by the system 100 shown in FIG. 1. The operations of the process 500 may be performed by the server 110 or the service station apparatus 130 in the system 100 shown in FIG. 1. The operations of the process 500 may be performed by the system 100 and the user device 140 shown in FIG. 1 cooperatively. The process 500 may be continued from the operation 206. An operation 501 may include: receiving a quantity of waiting people of a stop C. The communication module may be configured to perform the operation 501. In some embodiments, the quantity of waiting people of the stop A may indicate a quantity of people that wait for an ultrasound scan in a first ultrasound department. The quantity of waiting people of the stop C may indicate a quantity of people that wait for an ultrasound scan in a second ultrasound department. In some embodiments, the quantity of waiting people of the stop A may indicate a quantity of people that wait for paying a fine at a first payment counter. The quantity of waiting people of the stop C may indicate a quantity of people that wait for paying a fine at a second payment counter.

An operation 502 may include: determining a waiting time of the stop C The waiting time of the user at the stop C may be determined based on the quantity of waiting people of the stop C. The processing module may be configured to perform the operation 502. In some embodiments, the waiting time of the stop A may indicate a time for the patient to wait for the ultrasound scan in the first ultrasound department. The waiting time of the stop C may indicate a time for the patient to wait for the ultrasound scan in the second ultrasound department. In some embodiments, the waiting time of the stop A may indicate a time for the driver to wait for paying the fine at the first payment counter. The waiting time of the stop C may indicate a time for the driver to wait for paying the fine at the second payment counter.

An operation 503 may include: determining whether the waiting time of the stop A or the waiting time of the stop C is less than the queuing time of the service station P. The processing module may be configured to perform the operation 503. In some embodiments, the waiting time of the stop A or the waiting time of the stop C being less than the queuing time of the service station P may indicate that the waiting time of performing the ultrasound scan is less than the waiting time of seeing a doctor. In some embodiments, the waiting time of the stop A or the waiting time of the stop C being less than the queuing time of the service station P may indicate that the waiting time of paying the fine is less than the waiting time of replacing the driving license.

If the waiting time of the stop A or the waiting time of the stop C is less than the queuing time of the service station P, an operation 504 may be performed. The operation 504 may include: determining whether the waiting time of the stop C is less than the waiting time of the stop A. The processing module may be configured to perform the operation 504. In some embodiments, the waiting time of the stop C being less than the waiting time of the stop A may indicate that the waiting time of the second ultrasound department is less than the waiting time of the first ultrasound department. In some embodiments, the waiting time of the stop C being less than the waiting time of the stop A may indicate that the waiting time of the second payment counter is less than the waiting time of the first payment counter.

If the waiting time of the stop C is less than the waiting time of the stop A, an operation 505 may be performed. The operation 505 may include: queuing the user in a queue of the service station P. The processing module may be configured to perform the operation 505. In some embodiments, queuing the user in the queue of the service station P may indicate that the patient is queued in a queue of seeing a doctor. In some embodiments, queuing the user in the queue of the service station P may indicate that the driver is queued in a queue of replacing the driving license.

An operation 506 includes: queuing the user in a queue of the stop C. The processing module may be configured to perform the operation 506. In some embodiments, queuing the user in the queue of the stop C may indicate that the patient is queued in a queue of the second ultrasound department. In some embodiments, queuing the user in the queue of the stop C may indicate that the driver is queued in a queue of the second payment counter.

An operation 507 includes: transmitting a signal of going to the stop C to the user. The operation 507 includes: transmitting the signal of going to the stop C to the user device 140. The operation 507 includes: transmitting the signal of first going to the stop C and then going to the service station P to the user. The operation 507 includes: transmitting the signal of first going to the stop C and then going to the service station P to the user device 140. The communication module may be configured to perform the operation 507. In some embodiments, transmitting the signal of going to the stop C to the user may indicate that the patient is notified to go to the second ultrasound department. In some embodiments, transmitting the signal of going to the stop C to the user may indicate that the driver is notified to go to the second payment counter.

Referring to the operation 503 again, if the waiting time of the stop A or the waiting time of the stop C is not less than the queuing time of the service station P, it indicates that both the waiting time of the stop A and the waiting time of the stop C are greater than the queuing time of the service station P. In some embodiments, the waiting time of the stop A or the waiting time of the stop C being not less than the queuing time of the service station P may indicate that the waiting time of seeing a doctor is less than the waiting time of performing the ultrasound scan. In some embodiments, the waiting time of the stop A or the waiting time of the stop C being not less than the queuing time of the service station P may indicate that the waiting time of replacing the driving license is less than the waiting time of paying the fine. If the waiting time of the stop A or the waiting time of the stop C is not less than the queuing time of the service station P, the operation 211 may be performed.

Referring to the operation 504 again, if the waiting time of the stop C is not less than the waiting time of the stop A, the operation 208 may be performed. In some embodiments, the waiting time of the stop C being not less than the waiting time of the stop A may indicate that the waiting time of is the first ultrasound department is less than the waiting time of the second ultrasound department. In some embodiments, the waiting time of the stop C being not less than the waiting time of the stop A may indicate that the waiting time of the first payment counter is less than the waiting time of the second payment counter. In some embodiments, if the operation 210 is performed after the operation 504, transmitting the signal of going to the stop A to the user may indicate that the patient is notified to go to the first ultrasound department. In some embodiments, if the operation 210 is performed after the operation 504, transmitting the signal of going to the stop A to the user may indicate that the driver is notified to go to the first payment counter.

According to the process 500, the user may go to the stop A or the stop C according to an indication, to reduce the waiting time of the pre-work. The stop A and the stop C may be configured to provide a same service. The stop A and the stop C may be configured to process the same service.

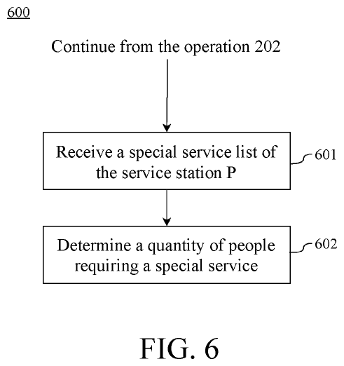

FIG. 6 shows a flowchart of a process 600 according to some embodiments of the present disclosure. Operations of the process 600 may be performed by the system 100 shown in FIG. 1. The operations of the process 600 may be performed by the server 110 or the service station apparatus 130 in the system 100 shown in FIG. 1. The operations of the process 600 may be performed by the system 100 and the user device 140 shown in FIG. 1 cooperatively. The process 600 may be continued from the operation 202. An operation 601 may include: receiving a special service list of the service station P. The communication module may be configured to perform the operation 601. In some embodiments, the special service list of the service station P may indicate a patient list that requires ambulatory surgery. In some embodiments, the special service list of the service station P may indicate a driver list that needs a barrier-free service. The ambulatory surgery may include, but not limited to, minimally invasive surgery and egg retrieval.

The operation 601 may include: determining a quantity of people requiring a special service. The operation 601 may include: determining the quantity of people requiring a special service that have not been served before the serial number of the user. The processing module may be configured to perform the operation 601. In some embodiments, it is assumed that a reservation serial number of a patient (for example, the serial number of the user data) is 20 and a current serial number of a consulting room (for example, the current serial number of the service station P) is 10, determining the quantity of people requiring a special service may indicate that a quantity of patients who require the ambulatory surgery (for example, the minimally invasive surgery or the egg retrieval) between No. 11 and No. 20 is determined. In some embodiments, it is assumed that a reservation serial number of a driver (for example, the serial number of the user data) is 20 and a current serial number of a driving license replacement counter (for example, the current serial number of the service station P) is 10, determining the quantity of people requiring a special service may indicate that a quantity of drivers that require the barrier-free service between No. 11 and No. 20.

In some embodiments, the operation 203 and other operations may be performed after the operation 601. An operation 203 may include: determining a queuing time of the service station P. If the operation 203 may be performed after the operation 601, the queuing time of the user in the service station P may be determined based on the current serial number of the service station P, the quantity of queued people of the service station P, the serial number of the user, and the quantity of people requiring a special service. In some embodiments, the queuing time of the service station P may indicate a time for a patient to wait for seeing a doctor. In some embodiments, the queuing time of the service station P may indicate a time for a driver to wait for replacing a driving license.

According to the process 600, the queuing time of the service station P may be more accurately determined based on the quantity of people that require a special service. According to the process 600, the user may be indicated to go to a specified stop based on the quantity of people that require the special service. According to the process 600, the user may be indicated to go to the specified stop earlier or later based on the quantity of people that require the special service. The special service may include, but not limited to, a process of the ambulatory surgery that needs to take a longer time than a general examination time. The special service may include, but not limited to, providing a priority serial number to the elder.

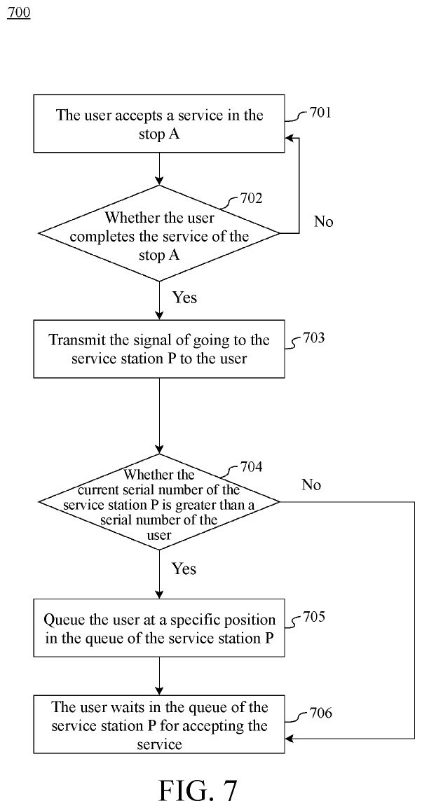

FIG. 7 shows a flowchart of a process 700 according to some embodiments of the present disclosure. Operations of the process 700 may be performed by the system 100 shown in FIG. 1. The operations of the process 700 may be performed by the server 110 or the service station apparatus 130 in the system 100 shown in FIG. 1. The operations of the process 700 may be performed by the system 100 and the user device 140 shown in FIG. 1 cooperatively. In some embodiments, the process 700 may be performed after the operation 210. An operation 701 may include: receiving, by the user, a service at the stop A. The processing module may be configured to perform the operation 701. In some embodiments, the receiving, by the user, the service at the stop A may indicate that a patient receives an ultrasound scan in an ultrasound department. In some embodiments, the receiving, by the user, the service at the stop A may indicate that a driver pays a fine in a payment counter.

An operation 702 may include: determining whether the user completes the service of the stop A. The processing module may be configured to perform the operation 702. In some embodiments, the user completing the service of the stop A may indicate that the patient has completed the ultrasound scan. In some embodiments, the user completing the service of the stop A may indicate that the driver has paid the fine.

If the user has not completed the service of the stop A, the operation 701 may be returned. If the user completes the service of the stop A, an operation 703 may be performed.

The operation 703 may include: transmitting the signal of going to the service station P to the user. The operation 703 may include: transmitting the signal of going to the service station P to the user device 140. The communication module may be configured to perform the operation 703. In some embodiments, the transmitting the signal of going to the service station P to the user may indicate that the patient is notified to go to a consulting room. In some embodiments, the transmitting the signal of going to the service station P to the user may indicate that the driver is notified to go to a driving license replacement counter.

An operation 704 may include: determining whether the current serial number of the service station P is greater than the serial number of the user. The processing module may be configured to perform the operation 704. In some embodiments, the current serial number of the service station P being greater than the serial number of the user may indicate that a serial number that is being in a consulting room is greater than a reservation serial number of the patient. For example, the reservation serial number of the patient is missed due to the ultrasound scan. In some embodiments, the current serial number of the service station P being greater than the serial number of the user may indicate that a serial number that is currently served by a driving license replacement counter is greater than a reservation serial number of the driver. For example, the reservation serial number of the driver is missed due to fine payment.

If the current serial number of the service station P is greater than the serial number of the user, an operation 705 may be performed. The operation 705 may include: queuing the user at a specific position of the queue of the service station P. The processing module may be configured to perform the operation 705. In some embodiments, it is assumed that a reservation serial number of a patient (for example, the serial number of the user data) is 20, a serial number that is being in a consulting room (for example, the current serial number of the service station P) is 23, and patients that currently check in the consulting room but not see a doctor include No. 24, No. 25, and No. 26, queuing the user at the specific position of the queue of the service station P may indicate that the patient with the reservation serial number of 20 is queued after the patient No. 25 and before the patient No. 26. In some embodiments, it is assumed that a reservation serial number of a driver (for is example, the serial number of the user data) is 20, a serial number that is being in a driving license replacement counter (for example, the current serial number of the service station P) is 23, and drivers that currently check in the driving license replacement counter but are not served include No. 24, No. 25, and No. 26, queuing the user at the specific position of the queue of the service station P may indicate that the driver with the reservation serial number of 20 is queued after the driver No. 24 and before the driver No. 25.

An operation 706 may include: waiting, by the user, for accepting the service in the queue of the service station P. The processing module may be configured to perform the operation 706. In some embodiments, the user waiting for accepting the service in the queue of the service station P may indicate that the patient waits for being called and seeing a doctor in a current queue of the consulting room. In some embodiments, the user waiting for accepting the service in the queue of the service station P may indicate that the driver waits for being called and accepting a service in a current queue of the driving license replacement counter.

In some embodiments, the operation 706 may be performed after the operation 705. In some embodiments, if the operation 706 is performed after the operation 705, the user waiting to accept the service in the queue of the service station P may indicate that the patient waits for being called and seeing the doctor in a queue set in the operation 705. In some embodiments, if the operation 706 is performed after the operation 705, the user waiting to accept the service in the queue of the service station P may indicate that the driver waits for being called and accepting the service in a queue set in the operation 705.

Reference is made to the operation 704 again. If the current serial number of the service station P is not greater than the serial number of the user, the operation 705 may be performed. In some embodiments, the current serial number of the service station P being not greater than the serial number of the user may indicate that the serial number that is being in the consulting room is not greater than the reservation serial number of the patient. For example, the reservation serial number of the patient does not miss due to the ultrasound scan. In some embodiments, the current serial number of the service station P being not greater than the serial number of the user may indicate that the serial number that is currently served by the driving license replacement counter is greater than the reservation serial number of the driver. For example, the reservation serial number of the driver does not miss due to the fine payment.

In some embodiments, the operation 706 may be performed after the operation 704. In some embodiments, if the operation 706 is performed after the operation 704, the user waiting for accepting the service in the queue of the service station P may indicate that the patient waits for being called and seeing a doctor in an originally set queue (for example, a queue set in the operation 208). In some embodiments, if the operation 706 is performed after the operation 704, the user waiting for accepting the service in the queue of the service station P may indicate that the driver waits for being called and accepting a service in an originally set queue (for example, a queue set in the operation 208).

According to the process 700, the user may first perform another pre-work according to an indication, to reduce the queuing time of the service station P. In addition, according to the process 700, if the user misses a serial number because of performing another pre-work according to an indication, a punishment waiting time of the user is reduced.

In some embodiments, all or some operations of the process 700 may be applicable to the embodiments in which the user first accepts the service in the service station P, then goes to the stop A to accept the service, and then returns to the service station P to accept the service. For example, a patient may first see a doctor in a consulting room, then goes to an ultrasound department to perform an ultrasound scan, and then returns to the consulting room to obtain evaluation and diagnosis from the doctor. According to the operation 705, the user may be queued at a specific position such as a first position or a second position in the queue of the service station P. For example, when the patient wants to return to the consulting room for an evaluation or diagnosis by a doctor, the patient may be queued at a first position (that is, a next patient), a second position, or another position in a queue.

According to some embodiments, one or more of the operation 704 to the operation 706 are applicable to the embodiment in which the user misses the serial number. For example, after a patient checks in, it may be determined, by using the operation 704, whether a current serial number of a consulting room is greater than a serial number of the patient. If the current serial number is greater than the serial number of the patient, it indicates that the patient misses the serial number, and the operation 705 and the operation 706 are performed. If the current serial number is not greater than the serial number of the patient, it indicates that the patient does not miss the serial number, and the operation 706 is performed. According to the operation 705, the user may be queued at a specific position such as a first position or a second position in the queue of the service station P. For example, the patient may be queued at a first position (that is, a next patient), a second position, or another position in a queue.

According to some embodiments, one or more of the operation 704 to the operation 706 are applicable to the embodiment in which the user is late for check-in. For example, when a suggested check-in time of a patient is 8:00 to 9:00 in the morning, and if the patient has not check in at 10:00 in the morning, it is determined that the patient misses a serial number due to late. In some embodiments, if it is determined that the user misses the serial number due to late for check-in, the operation 705 and the operation 706 may be performed. According to the operation 705, the user may be queued at a specific position such as a first position or a second position in the queue of the service station P. For example, the patient may be queued at a first position (that is, a next patient), a second position, or another position in a queue.

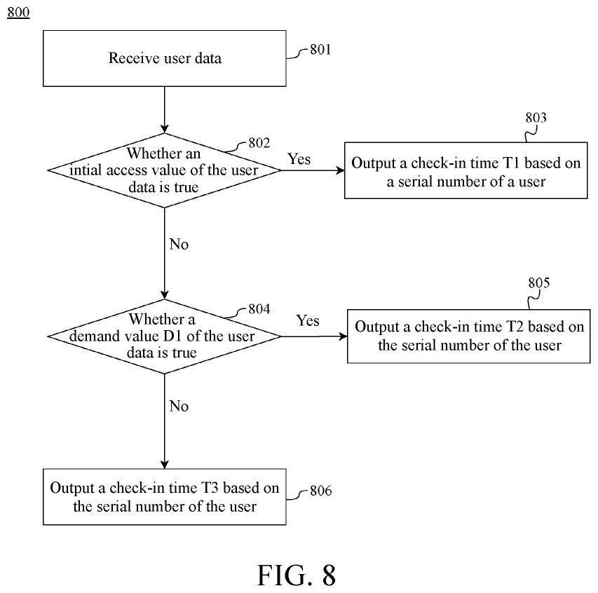

FIG. 8 shows a flowchart of a process 800 according to some embodiments of the present disclosure. Operations of the process 800 may be performed by the system 100 shown in FIG. 1. The operations of the process 800 may be performed by the server 110 or the service station apparatus 130 in the system 100 shown in FIG. 1. The operations of the process 800 may be performed by the system 100 and the user device 140 shown in FIG. 1 cooperatively. The process 800 may be performed when the user makes an appointment with an organization such as a hospital, a clinic, a bank, or a motor vehicles office. An operation 801 may include: receiving user data. The user data may be received from the database 120 or inputted by the user in a reservation interface. The communication module may be configured to perform the operation 801. In some embodiments, the operation 801 may be performed in a case that the user inputs basic data or reservation data in the organization such as the hospital, the clinic, the bank, or the motor vehicles office.

An operation 802 may include: determining whether an initial access value of the user data is true. The initial access value of the user data may be a column in the user data. The processing module may be configured to perform the operation 802. In some embodiments, the initial access value of the user data being true may indicate that the patient does not go to the hospital or clinic for seeing a doctor. In some embodiments, the initial access value of the user data being true may indicate that the driver has never replaced the driving license.

If the initial access value of the user data is true, an operation 803 may be performed. The operation 803 may include: outputting a check-in time Ti based on the serial number of the user. The serial number of the user may be at a serial number column of the user data. The processing module and/or the communication module may be configured to perform the operation 803. The outputting the check-in time Ti may include outputting the check-in time T1 to the user device 140 by using text or sound. In some embodiments, the serial number of the user data may indicate a reservation serial number allocated to the patient after the patient inputs data in a hospital or clinic reservation interface. In some embodiments, the serial number of the user data may indicate a reservation serial number allocated to the driver after the driver inputs data in a motor vehicles office reservation interface. In some embodiments, it is assumed that the serial number of the user is 23 (for example, the reservation serial number is 23), an estimated check-in time may be 10:30 in the morning. However, because the initial access value is true (for example, a first visit or a new customer), the outputted check-in time T1 may be 10:10 in the morning (that is, 20 minutes earlier), to reserve time for related operations (for example, creating new patient data or creating new customer data).

If the initial access value of the user data is not true, an operation 804 may be performed. The operation 804 may include: determining whether the demand value D1 of the user data is true. The demand value D1 of the user data may be a column in the user data. The processing module may be configured to perform the operation 804. In some embodiments, the demand value D1 of the user being true may indicate that the patient needs to be first checked (for example, an ultrasound scan or blood examination) before seeing a doctor. In some embodiments, the demand value D1 of the user being true may indicate that the driver needs to first go through another procedure (for example, paying a fine or performing a cognitive function test) before replacing a driving license.

If the demand value D1 of the user data is true, an operation 805 may be performed. The operation 805 may include: outputting a check-in time T2 based on the serial number of the user. The serial number of the user may be at a serial number column of the user data. The processing module and/or the communication module may be configured to perform the operation 805. The outputting the check-in time T2 may include outputting the check-in time T2 to the user device 140 by using text or sound. In some embodiments, it is assumed that the serial number of the user is 23 (for example, the reservation serial number is 23), an estimated check-in time may be 10:30 in the morning. However, because the demand value D1 is true (for example, another check or procedure is first performed), the outputted check-in time T2 may be 10:00 in the morning (that is, 30 minutes earlier), to reserve time for another check or procedure.

If the demand value D1 of the user data is not true, an operation 806 may be performed. The operation 806 may include: outputting a check-in time T3 based on the serial number of the user. The serial number of the user may be at a serial number column of the user data. The processing module and/or the communication module may be configured to perform the operation 806. The outputting the check-in time T3 may include outputting the check-in time T3 to the user device 140 by using text or sound. In some embodiments, it is assumed that the serial number of the user is 23 (for example, the reservation serial number is 23), an estimated check-in time may be 10:30 in the morning. However, because neither the initial access value nor the demand value D1 is true, the outputted check-in time T3 may be 10:30 in the morning.

According to the process 800, different check-in times may be allocated to users who reserve in a same day, to achieve a diversion effect. According to the process 800, different check-in times may be allocated to a plurality of users in a single day. According to the process 800, the user may complete check-in at a specified time point according to an indication.

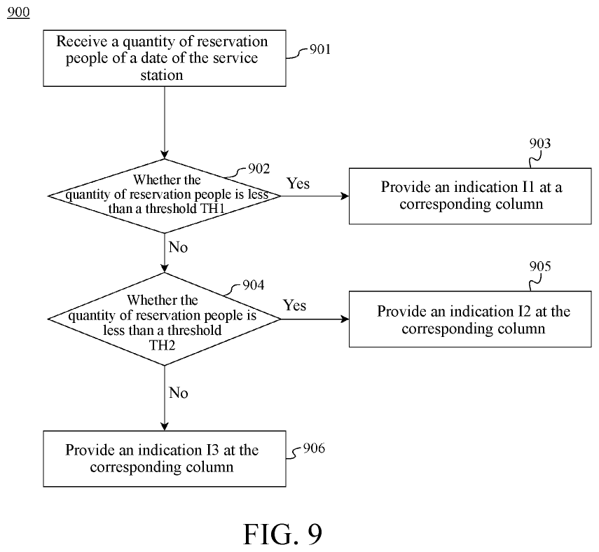

FIG. 9 shows a flowchart of a process 900 according to some embodiments of the present disclosure. Operations of the process 900 may be performed by the system 100 shown in FIG. 1. The operations of the process 900 may be performed by the server 110 or the service station apparatus 130 in the system 100 shown in FIG. 1. The operations of the process 900 may be performed by the system 100 and the user device 140 shown in FIG. 1 cooperatively. The process 900 may be performed when the user makes an appointment with an organization such as a hospital, a clinic, a bank, or a motor vehicles office. An operation 901 may include: receiving a quantity of reservation people of a date of the service station P. The operation 901 may include: receiving a quantity of known reservation people of the date of the service station P. The quantity of reservation people of the date of the service station P may be received from the database 120. The communication module may be configured to perform the operation 901. In some embodiments, the operation 901 may be performed in a case that the user inputs basic data or reservation data in the organization such as the hospital, the clinic, the bank, or the motor vehicles office. In some embodiments, the quantity of reservation people of the data of the service station P may indicate a quantity of people who have made appointments with a doctor Li in the morning.

An operation 902 may include: determining whether the quantity of reservation people is less than a threshold TH1. The operation 902 may include: determining whether the quantity of reservation people received in the operation 901 is less than the threshold TH1. The processing module may be configured to perform the operation 902. In some embodiments, determining whether the quantity of reservation people is less than the threshold TH1 may indicate that it is determined whether the quantity of people who have made appointments with the doctor Li in the morning is less than 10. It should be noted that TH1 not only depends on the quantity of people, but also depends on another parameter. For example, TH1 may depend on a quantity of required service items known to a reserved user. For example, TH1 may depend on a condition such as a device or a resource on a day (for example, the stops or the service stations are reduced due to a condition such as a device, a resource, or a person). TH1 may not be fixed. TH1 may be fixed.

If the quantity of reservation people is less than the threshold TH1, an operation 903 may be performed. The operation 903 may include: providing an indication I1 at a corresponding column. The operation 903 may include: providing the indication I1 at a column corresponding to the data indicated in the operation 901. The corresponding column may indicate information such as a date or a place. The corresponding column may present the information such as the date or the place. The processing module and/or the communication module may be configured to perform the operation 903. The providing the indication I1 may include: providing the indication I1 to the user device 140 by using a color, text, or sound. In some embodiments, providing the indication I1 at the corresponding column may indicate that an indication of a color (for example, green), text (for example, “empty”), or sound is provided at a corresponding column of a doctor Li in the morning one day. In some embodiments, providing the indication I1 at the corresponding column may indicate that an indication of a color (for example, green), text (for example, “empty”), or sound is provided at a corresponding column in the morning one day.

Referring to the operation 902 again, if the quantity of reservation people is not less than the threshold TH1, an operation 904 may be performed. An operation 904 may include: determining whether the quantity of reservation people is less than a threshold TH2. The operation 904 may include: determining whether the quantity of reservation people received in the operation 901 is less than the threshold TH2. The processing module may be configured to perform the operation 904. In some embodiments, determining whether the quantity of reservation people is less than the threshold TH2 may indicate that it is determined whether the quantity of people who have made appointments with the doctor Li in the morning is less than 30. It should be noted that TH2 not only depends on the quantity of people, but also depends on another parameter. For example, TH2 may depend on a quantity of required service items known to a reserved user. For example, TH2 may depend on a condition such as a device or a resource on a day (for example, the stops or the service stations are reduced due to a condition such as a device, a resource, or a person). TH2 may not be fixed. TH2 may be fixed.

If the quantity of reservation people is less than the threshold TH2, an operation 905 may be performed. The operation 905 may include: providing an indication I2 at a corresponding column. The corresponding column may indicate information such as a date or a place. The corresponding column may present the information such as the date or the place. The operation 905 may include: providing the indication I2 at a column corresponding to the data indicated in the operation 901. The processing module and/or the communication module may be configured to perform the operation 905. The providing the indication I2 may include: providing the indication I2 to the user device 140 by using a color, text, or sound. In some embodiments, providing the indication I2 at the corresponding column may indicate that an indication of a color (for example, yellow), text (for example, “busy”), or sound at the corresponding column of the doctor Li in the morning one day. In some embodiments, providing the indication I2 at the corresponding column may indicate that an indication of a color (for example, yellow), text (for example, “busy”), or sound is provided at the corresponding column in the morning one day.

Referring to the operation 904 again, if the quantity of reservation people is not less than the threshold TH2, an operation 906 may be performed. The operation 906 may include: providing an indication I3 at a corresponding column. The corresponding column may indicate information such as a date or a place. The corresponding column may present the information such as the date or the place. The operation 906 may include: providing the indication I3 at a column corresponding to the data indicated in the operation 901. The processing module and/or the communication module may be configured to perform the operation 906. The providing the indication I3 may include: providing the indication I3 to the user device 140 by using a color, text, or sound. In some embodiments, providing the indication I3 at the corresponding column may indicate that an indication of a color (for example, red), text (for example, “full”), or sound at the corresponding column of the doctor Li in the morning one day. In some embodiments, providing the indication I3 at the corresponding column may indicate that an indication of a color (for example, red), text (for example, “full”), or sound is provided at the corresponding column in the morning one day.

According to the process 900, effects of diversion and averaging a quantity of reservation people on different days can be achieved. According to the process 900, different reservation dates may be offered to a plurality of users.

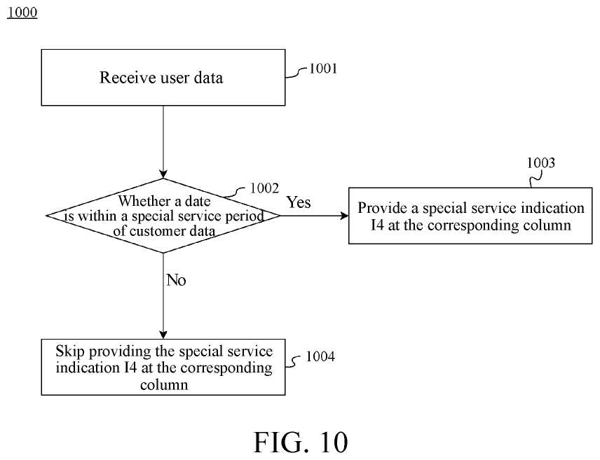

FIG. 10 shows a flowchart of a process 1000 according to some embodiments of the present disclosure. Operations of the process 1000 may be performed by the system 100 shown in FIG. 1. The operations of the process 1000 may be performed by the server 110 or the service station apparatus 130 in the system 100 shown in FIG. 1. The operations of the process 1000 may be performed by the system 100 and the user device 140 shown in FIG. 1 cooperatively. The process 1000 may be performed when the user makes an appointment with an organization such as a hospital, a clinic, a bank, or a motor vehicles office. An operation 1001 may include: receiving user data. The user data may be received from the database 120. The communication module may be configured to perform the operation 1001.

An operation 1002 may include: determining whether a date is within a special service period of customer data. The special service period may be a column used for indicating a period in the customer data. For example, a column of customer data may include “20210406 to 20210413”, to indicate a special service period. The processing module may be configured to perform the operation 1002. In some embodiments, determining whether the date is within the special service period of the customer data may indicate that it is determined whether a date is within an ovulation period of a patient.

If it is determined that the date is within the special service period of the customer data, an operation 1003 may be performed. The operation 1003 may include: providing a special service indication I4 at the corresponding column. The corresponding column may indicate information such as a date or a place. The corresponding column may present the information such as the date or the place. The operation 1003 may include: providing the indication I4 at a column corresponding to the data indicated in the operation 1002. The processing module and/or the communication module may be configured to perform the operation 1003. The providing the indication I4 may include: providing the indication I4 to the user device 140 by using a color, text, or sound. In some embodiments, providing the indication I4 at the corresponding column may indicate that an indication of a color (for example, pink), text (for example, “ovulation period”), or sound is provided at a corresponding column of Apr. 6, 2021 to Apr. 13, 2021.

If it is determined that the date is not within the special service period of the customer data, an operation 1004 may be performed. The operation 1004 may include: skipping providing the special service indication I4 at the corresponding column. The operation 1004 may include: skipping providing the indication I4 at the column corresponding to the data indicated in the operation 1002. The processing module and/or the communication module may be configured to perform the operation 1004. In some embodiments, skipping providing the indication I4 at the corresponding column may indicate that the indication of a color (for example, pink), text (for example, “ovulation period”), or sound is not provided at the corresponding column of Apr. 6, 2021 to Apr. 13, 2021.

According to the process 1000, a service may be reserved according to the special service period of the user (for example, the ovulation period), and a related service (for example, minimally invasive surgery or egg retrieval) may be smoothly performed.

Although the present disclosure has been described with reference to the descriptions of the specific embodiments of the present disclosure, this description is not intended to limit the present disclosure. A person skilled in the art should understand that modifications and alternative equivalents can be made without departing from the scope and spirit of the present disclosure as defined in the appended claims. The description may not be drawn to scale. Due to manufacturing processes and tolerances, differences may exist between the artistic representation in this application and the artistic representation in the actual disclosure. There may be other embodiments of the present disclosure that are not specifically described. The specification and the accompanying drawings should be considered as illustrative but not restrictive. Modifications may be made, so that a specific situation, material, a composition of an object, a method, or a process is applicable to the objective, the spirit, and the scope of the present disclosure. All the modifications are intended to be within the scope of the claims attached herein. Although the method disclosed in the specification is described with reference to the specific operations which are performed in a specific sequence, it should be understood that those operations can be combined, subdivided or re-sequenced to form equivalent methods without departing from the instruction of the present disclosure. Therefore, unless otherwise specified, the sequence and grouping of the operations do not limit the present disclosure. In addition, the effects detailed in the embodiments and the like are merely examples. Therefore, this application may further have another effect.

In addition, the logic processes depicted in the accompanying drawings are not necessarily performed in the specific order or successively to achieve an expected result. In addition, other steps may be provided, or a plurality of steps may be removed from the process, and another component may be added to or removed from the system. Therefore, other embodiments are all within the scope of the appended claims.

What is claimed is:

1. An electronic device, comprising:
a communication module, configured to be communicatively coupled to a user device of a user, wherein the communication module is configured to:
receive user data of the user;
receive a current serial number and a quantity of queued people of a service station;
in response to a first demand value of the user data being true, receive a quantity of waiting people of a first stop; and
in response to a first waiting time being less than a queuing time, transmit a first signal of going to the first stop to the user device;
a memory module, configured to store a plurality of instructions and information; and
a processing module, configured to be coupled to the communication module and the memory module and perform the following operations based on the instructions and the information stored in the memory module:
determining the queuing time based on the current serial number of the service station, the quantity of queued people of the service station, and a serial number of the user data;
in response to the first demand value of the user data being true, determining the first waiting time based on the quantity of waiting people of the first stop; and
in response to the first waiting time being less than the queuing time, queuing the user in a queue of the service station and a queue of the first stop.

2. The electronic device according to claim 1, wherein the communication module is further configured to:
in response to a second demand value of the user data being true, receive a quantity of waiting people of a second stop; and
the processing module is further configured to:
in response to the second demand value of the user data being true, determine a second waiting time based on the quantity of waiting people of the second stop; and
in response to a sum of the first waiting time and the second waiting time being less than the first queuing time, queue the user in the queue of the service station, the queue of the first stop, and a queue of the second stop.

3. The electronic device according to claim 2, wherein the communication module is further configured to:
in response to the first waiting time being less than the second waiting time, transmit the first signal of going to the first stop to the user device.

4. The electronic device according to claim 2, wherein the communication module is further configured to:
in response to the first waiting time being not less than the second waiting time, transmit a second signal of going to the second stop to the user device.

5. The electronic device according to claim 1, wherein the communication module is further configured to:
in response to the first demand value of the user data being true, receive a quantity of waiting people of a third stop; and
in response to a third waiting time being less than the queuing time and the third waiting time being less than the first waiting time, transmit a third signal of going to the third stop to the user device; and
the processing module is further configured to:
in response to the first demand value of the user data being true, determine the third waiting time based on the quantity of waiting people of the third stop; and
in response to the third waiting time being less than the queuing time and the third waiting time being less than the first waiting time, queue the user in the queue of the service station and a queue of the third stop.

6. The electronic device according to claim 1, wherein the communication module is further configured to receive a special service list of the service station; and
the processing module is further configured to:
determine a quantity of people requiring a special service based on the current serial number of the service station, the serial number of the user data, and the special service list; and
determine the queuing time based on the current serial number of the service station, the quantity of queued people of the service station, the serial number of the user data, and the quantity of people requiring a special service.

7. The electronic device according to claim 1, wherein the communication module is further configured to, in response to that the first stop has completed a service of the user, transmit a fourth signal of going to the service station to the user device; and
the processing module is further configured to, in response to the current serial number of the service station being greater than the serial number of the user data, queue the user at a special position of the queue of the service station.

8. The electronic device according to claim 1, wherein the communication module is further configured to:
in response to an initial access value of the user data being true, output a first check-in time based on the serial number of the user data;
in response to the first demand value of the user data being true, output a second check-in time based on the serial number of the user data; and
in response to the initial access value of the user data being not true and the first demand value of the user data being not true, output a third check-in time based on the serial number of the user data.

9. The electronic device according to claim 1, wherein the communication module is further configured to:
receive a quantity of reservation people of the service station; and
provide an indication at a column of the user device based on the quantity of reservation people of the service station, wherein the providing the indication at the column of the user device comprises:
in response to the quantity of reservation people of the service station being less than a first threshold, providing a first indication at the column of the user device;
in response to the quantity of reservation people of the service station being not less than the first threshold and being less than a second threshold, providing a second indication at the column of the user device; and
in response to the quantity of reservation people of the service station being not less than the second threshold, providing a third indication at the column of the user device.

10. The electronic device according to claim 1, wherein the communication module is further configured to:
in response to a special service period of the user data, provide a special service indication at a column of the user device.

11. A method for queuing, comprising:
receiving user data of a user;
receiving a current serial number and a quantity of queued people of a service station;
determining a queuing time based on the current serial number, the quantity of queued people, and a serial number of the user data;
in response to a first demand value of the user data being true, receiving a quantity of waiting people of a first stop;
determining a first waiting time based on the quantity of waiting people of the first stop; and
in response to the first waiting time being less than the queuing time, transmitting a first signal of going to the first stop to the user, and queuing the user in a queue of the service station and a queue of the first stop.

12. The method according to claim 11, further comprising:
in response to a second demand value of the user data being true, receiving a quantity of waiting people of a second stop;
determining a second waiting time based on the quantity of waiting people of the second stop; and
in response to a sum of the first waiting time and the second waiting time being less than the queuing time, queuing the user in the queue of the service station, the queue of the first stop, and a queue of the second stop.

13. The method according to claim 12, further comprising:
in response to the first waiting time being less than the second waiting time, transmitting the first signal of going to the first stop to the user.

14. The method according to claim 12, further comprising:
in response to the first waiting time being not less than the second waiting time, transmitting a second signal of going to the second stop to the user.

15. The method according to claim 11, further comprising:
in response to the first demand value of the user data being true, receiving a quantity of waiting people of a third stop;
determining a third waiting time based on the quantity of waiting people of the third stop; and
in response to the third waiting time being less than the queuing time and the third waiting time being less than the first waiting time, transmitting a third signal of going to the third stop to the user, and queuing the user in the queue of the service station and a queue of the third stop.

16. The method according to claim 11, further comprising:
receiving a special service list of the service station;
determining a quantity of people requiring a special service based on the current serial number of the service station, the serial number of the user data, and the special service list; and
determining the queuing time based on the current serial number of the service station, the quantity of queued people of the service station, the serial number of the user data, and the quantity of people requiring a special service.

17. The method according to claim 11, further comprising:
in response to that the first stop has completed a service of the user, transmitting a fourth signal of going to the service station to the user; and
in response to the current serial number of the service station being greater than the serial number of the user data, queuing the user at a special position of the queue of the service station.

18. The method according to claim 11, further comprising:
in response to an initial access value of the user data being true, outputting a first check-in time based on the serial number of the user data;
in response to the first demand value of the user data being true, outputting a second check-in time based on the serial number of the user data; and
in response to the initial access value of the user data being not true and the first demand value of the user data being not true, outputting a third check-in time based on the serial number of the user data.

19. The method according to claim 11, further comprising:
receiving a quantity of reservation people of the service station; and
providing an indication at a column based on the quantity of reservation people of the service station, wherein
the providing the indication at the column comprises:
in response to the quantity of reservation people of the service station being less than a first threshold, providing a first indication at the column;
in response to the quantity of reservation people of the service station being not less than the first threshold and being less than a second threshold, providing a second indication at the column; and
in response to the quantity of reservation people of the service station being less than the second threshold, providing a third indication at the column.

20. The method according to claim 11, further comprising:
in response to a special service period of the user data, providing a special service indication at a column.
